(19) United States
(12) Patent Application Publication
(10) Pub. No.: US 2020/0370011 A1
(43) Pub. Date: Nov. 26, 2020

(54) GERIATRIC CAR-T CELLS AND USES THEREOF

(71) Applicant: PROSPECT CHARTERCARE RWMC, LLC d/b/a ROGER WILLIAMS MEDICAL CENTER, Providence, RI (US)

(72) Inventor: Steven C. KATZ, Providence, RI (US)

(21) Appl. No.: 16/068,063

(22) PCT Filed: Jan. 6, 2017
(86) PCT No.: PCT/US2017/012549

§ 371 Date: Jul. 3, 2018

Related U.S. Application Data
(60) Provisional application No. 62/276,693, filed on Jan. 8, 2016.

(51) Int. Cl. C12N 5/0783; C12N 15/86; A61K 35/17; C07K 14/725; C07K 16/30; C07K 14/705; A61P 35/00

(52) U.S. Cl. CPC C12N 5/0636; C12N 15/86; A61K 35/17; C07K 14/7051; C07K 16/3007; C07K 2319/03; A61P 35/00; C12N 2501/22; C12N 2501/15; C12N 2510/00; C07K 14/70517

(57) ABSTRACT

Chimeric antigen receptors (CARs) expressing T cells are a promising form of immunotherapy for solid tumors. CAR-T cells from geriatric donors (gCART) are shown herein to be functionally impaired relative to CAR-T from younger donors (yCAR-T). Higher transduction efficiencies and improved cell expansion were observed in yCAR-T cells compared to gCAR-T. yCAR-T demonstrated significantly increased levels of proliferation and signaling activation of pERK, pAKT, pSTAT3 and pSTAT5. Furthermore, yCAR-T contained higher proportions of CD4 and CD8 effector memory cells (EM) which are known to have enhanced cytolytic capabilities. In accordance with higher numbers of CD4 and CD8 EM, yCAR-T demonstrated higher levels of CEA specific cytotoxicity compared to gCAR-T, with maximum cytotoxicity observed in IL15 treated yCAR-T cells.

### TECHNICAL FIELD

The subject matter described herein relates to methods for increasing the efficiency of transducing ex vivo peripheral blood mononuclear cells (PBMC) with a recombinant virus which harbors a recombinant DNA molecule that encodes a chimeric antigen receptor (CAR) construct. The PBMC can be harvested from geriatric subjects suffering from a disease such as cancer, transduced with a CAR of interest and administered back to the subject for treatment of the disease.

### BACKGROUND

Cancer is a disease of elderly people, the median age being 70 years in industrialized countries (Gloeckler et al., 2003, Oncologist, 8:541-552). The elderly have accumulated more genetic damage related to environmental carcinogens and have reduced immune function or “immunosenescence” (Gloeckler et al., 2003, Oncologist, 8:541-552). Immunosenescence is characterized by a contraction of the naïve T cell compartment and CD4+ and CD8+ T cell functional deficiencies, which impair anti-tumor immunity (Naylor et al., 2005, J Immunol, 174:7446-7452). As such, cancer immunotherapy must account for age-related immune changes.

There has been significant progress in the field of adoptive T cell therapy (ACT), primarily through development of CAR-T cell platforms. Autologous T cells can be genetically engineered to express proteins that confer exquisite specificity for tumor antigens by introducing genes that encode synthetic chimeric antigen receptors (CARs). In a recent Phase I HITM trial and ongoing HITM-SIR Phase Ib study, we tested the safety of the CAR-T cells that are specific for carcinoembryonic antigen (CEA) are being tested. CEA is expressed in several types of cancers including but not limited to colorectal cancer, breast cancer, prostate cancer, and lung cancer. The infusion of CAR-T cells that are tumor antigen specific offers patients an immediate highly specific immune response, to contrast to vaccines which attempt to generate immunity within the host.

In elderly patients there is a major phenotypic shift from naïve to memory effector T cell population that could render the ACT not as effective as treating young patients (Kovaiou et al., 2005, Int Immunol 17:1359-1366). Interleukin-2 (IL2) and interleukin-15 (IL15) are known to signal through the common gamma chain (yc) and the IL2 β chain receptors and are critical but functionally distinct regulators of T cell proliferation and differentiation. Both IL2 and IL15 are known to activate the Jak/Stat, PI3k/Akt and Mek/Erk signaling pathways (Marzec et al., 2008, Cancer Res, 68:1083-1091) but they have differential immunotherapeutic effects. IL15 has superior effect on generating memory and effector T cells and hence there is a growing trend in using exogenous IL15 instead of IL2 for immunotherapies (Waldmann, 2006, Nat Rev Immunol, 6:595-601).

The anti-tumor activity of CAR-T cells generated from geriatric donors was studied and experiments were performed to evaluate whether IL2 and IL15 had any differential effects on the CAR-T phenotypes in both geriatric and young donors. The results, provided in the present disclosure, show that T cells obtained from geriatric donors are transduced at a lower efficiency compared to cells from younger donors, resulting in lower cytotoxicity and tumor killing activity of the geriatric T cells. However, as shown in the present disclosure, transduction efficiency of geriatric T-cells can be increased by treating harvested cells with agents which increase expressed of α5β1. The foregoing examples of the related art and limitations related therewith are intended to be illustrative and not exclusive. Other limitations of the related art will become apparent to those of skill in the art upon a reading of the specification and a study of the drawings.

### BRIEF SUMMARY

The following aspects and embodiments thereof described and illustrated below are meant to be exemplary and illustrative, not limiting in scope.

In one aspect, a method for transducing a population of lymphocytes ex vivo is provided, the method comprising contacting ex vivo the lymphocytes with an agent which increases expression of α5β1 by the lymphocytes and mixing the lymphocytes with a recombinant viral particle, wherein the recombinant viral vector harbors a recombinant DNA molecule encoding a chimeric antigen receptor (CAR). In some embodiments, the method increases efficiency of transduction of the lymphocytes by the recombinant viral particle as compared to the efficiency of transduction of lymphocytes which were not contacted with an agent that increases expression of α5β1.

In some embodiments, the population of lymphocytes comprises T cells, B cells and/or NK cells. In other embodiments, the T cells comprise CD4+ cells, CD8+ cells, gamma delta T cells (γδ T cells), NK T cells and/or regulatory T cells (Treg).

In some embodiments, the agent which increases expression of α5β1 comprises macrophage colony stimulating factor (M-CSF) or transforming growth factor beta-1 (TGFβ1). In other embodiments, the agent which increases expression of α5β1 comprises M-CSF and TGFβ1.

In some embodiments, the agent increases expression of α5β1 by the population of lymphocytes by at least about 5%, 10%, 20%, 30%, 40%, 50%, 60%, 70%, 80% or 90% of the expression of α5β1 by an equivalent population of lymphocytes which are not transduced with the agent. In other embodiments, the increase in expression of M-CSF and/or TGFβ1 is measured by quantitating mRNA which encodes M-CSF and/or TGFβ1. In still other embodiments, the increase in expression of M-CSF and/or TGFβ1 is measured by quantitating M-CSF and/or TGFβ1 protein levels. In some embodiments, one or more cells within the population of lymphocytes does not express endogenous M-CSF and TGFβ1 protein.

In some embodiments, the contacting the lymphocytes with the agent comprises incubating about 1×10⁵ to 1×10⁸, 1×10⁶ to 1×10⁷, 1×10⁶ to 1×10⁸ lymphocytes with the agent. In other words, in some embodiments, the population of lymphocytes comprises about 1×10⁵ to 1×10⁸, 1×10⁶ to 1×10⁷, 1×10⁶ to 1×10⁸ lymphocytes. In other embodiments, the contacting the lymphocytes with the agent comprises incubating about 1×10⁵, 1×10⁶ 1×10⁷ or 1×10⁸ lymphocytes with the agent.

In some embodiments, the contacting the cells with M-CSF comprises incubating the cells with 0.1 ng/ml to 10 ng/ml, 2.5 to 7.5 nm/ml or 4 ng/ml to 7 ng/ml M-CSF.

In some embodiments, the contacting the cells with TGFβ1 comprises incubating the cells with 1 ng/ml to 20 ng/ml, 5 ng/ml to 15 nm/ml, or 7.5 ng/ml to 12.5 ng/ml TGFβ1.

In some embodiments, prior to contacting the cells with M-CSF or TGFβ1 the cells are obtained from a subject diagnosed with a disease. In other embodiments, the disease is a cancer or immune deficiency disease. In still other embodiments, the cancer is selected from the group consisting of a liver cancer, a pancreatic cancer, a leukemia, a lymphatic cancer, a brain cancer, a head & neck cancer, a lung cancer, a breast cancer, a thyroid cancer, a prostate cancer, a stomach cancer, an esophageal cancer, a colon cancer, a rectal cancer, a testicular cancer, a bladder cancer, a cervical cancer, an ovarian cancer and a skin cancer. In yet other embodiments, the immune deficiency disease is caused by the human immunodeficiency virus (HIV).

In some embodiments, the recombinant viral particle is a lentiviral, retroviral, adenoviral or adeno-associated viral vector.

In some embodiments, the CAR encodes a T-cell receptor which binds to a tumor antigen. In other embodiments, the tumor antigen is selected from the group consisting of In still other embodiments, the tumor antigen is carcinoembryonic antigen (CEA).

In another aspect, a method for treating a subject in need thereof is provided wherein the method comprises administering to the subject a composition comprising a CAR-T generated using any of the methods described herein.

In some embodiments, the subject is at least 55 years old, 60 years old, 65 years old, 70 years old or 75 years old.

In some embodiments, the subject has been diagnosed with a cancer. In other embodiments, the cancer is selected from the group consisting of a liver cancer, a pancreatic cancer, a leukemia, a lymphatic cancer, a brain cancer, a head & neck cancer, a lung cancer, a breast cancer, a thyroid cancer, a prostate cancer, a stomach cancer, an esophageal cancer, a colon cancer, a rectal cancer, a testicular cancer, a bladder cancer, a cervical cancer, an ovarian cancer and a skin cancer.

Additional embodiments of the present methods and compositions, and the like, will be apparent from the following description, drawings, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment of the present invention. Additional aspects and advantages of the present invention are set forth in the following description and claims, particularly when considered in conjunction with the accompanying examples and drawings.

### BRIEF DESCRIPTION OF DRAWINGS

FIG. 1A illustrates gated flow cytometry of PBMCs isolated from a human subject then transduced with a recombinant viral vector harboring a construct encoding a CAR.

FIG. 1B shows a graph of viral transduction efficiency of geriatric and young PBMC (n=8; *p=0.002).

FIGS. 2A and 2B show graphs of untransduced (FIG. 2A) and transduced (FIG. 2B) geriatric and young cells in the presence or absence of IL2 or IL15. (* p<0.05 geriatric vs. young for respective treatments)

FIGS. 3A and 3B show expression levels of STAT3, STATS, Erk and Akt in geriatric or young cells by western blot analysis (*p<0.05 geriatric vs. young for respective treatments).

FIGS. 4A and 4B show CEA-specific cell cytotoxicity in the presence of transduced (FIG. 4A) or untransduced (FIG. 4B) young or geriatric cells that had been maintained in the presence of IL2 or IL15 (* p<0.005 geriatric vs. young for respective treatments).

FIG. 5A shows granzyme B levels in culture media in which young or geriatric anti-CEA CAR-T cells were maintained in either IL2 or IL15 prior to incubation with tumor cells (*p<0.005 geriatric vs. young for respective treatments).

FIG. 5B shows preforin levels in culture media in which young or geriatric anti-CEA CAR-T cells were maintained in either IL2 or IL15 prior to incubation with tumor cells (* p<0.05 geriatric vs. young for respective treatments). (*p<0.005 geriatric vs. young for respective treatments).

FIG. 6A illustrates gated flow cytometry of PBMCs isolated from young and geriatric human subjects and transduced with a recombinant viral vector harboring a construct encoding a CAR.

FIGS. 6B, 6C, and 6D show graphs of T cell populations in transduced young and geriatric CAR T cells. Relative levels are shown of CD4 effector memory cells (FIG. 6B; *p<0.05 geriatric vs. young for respective treatments), CD4 effector cells (FIG. 6C; *p<0.05 geriatric vs. young for respective treatments) and CD8 effector memory cells (FIG. 6D; *p<0.05 vs. corresponding geriatric donor cells).

FIG. 7A illustrates gated flow cytometry of PBMCs isolated from young and geriatric human subjects, treated with M-CSF or TGFβ1, and transduced with a recombinant viral vector harboring a construct encoding a CAR.

FIGS. 7B and 7C show graphs of α5β1 integrin expression (FIG. 7B; *p<0.0005 vs. geriatric) and CAR expression (FIG. 7C; *p<0.005 vs. geriatric) in young and geriatric cells.

FIGS. 8A and 8B show graphs of cytotoxicity of young and geriatric CAR-T cells which had been expanded and maintained in IL2 (FIG. 8A; *p<0.0005 vs. geriatric) or IL15 (FIG. 8B; *p<0.0005 vs. geriatric) then treated with M-CSF or TGFβ1.

### DETAILED DESCRIPTION

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

### I. Definitions

As used in this specification, the singular forms “a,” “an,” and “the” include plural referents unless the context clearly dictates otherwise. Thus, for example, reference to a “polymer” includes a single polymer as well as two or more of the same or different polymers, reference to an “excipient” includes a single excipient as well as two or more of the same or different excipients, and the like.

Where a range of values is provided, it is intended that each intervening value between the upper and lower limit of that range and any other stated or intervening value in that stated range is encompassed within the disclosure. For example, if a range of 1 μm to 8 μm is stated, it is intended that 2 μm, 3 μm, 4 μm, 5 μm, 6 μm, and 7 μm are also explicitly disclosed, as well as the range of values greater than or equal to 1 μm and the range of values less than or equal to 8 μm.

“macrophage colony stimulating factor” or “M-CSF” as used herein refers to a protein described, for example, in GenBank record Accession Number NP 000748, and optionally any precursors, variants or isoforms thereof.

“Transforming growth factor beta-1” or “TGFβ1” as used herein refers to a protein described, for example, in GenBank record Accession Number NP 000651, and optionally any precursors, variants or isoforms thereof.

“Geriatric donor” as used herein refers to a human who at least 55 years, 60 years, 65 years, 70 years, or 75 years of age. “Geriatic T cells” as used herein refers to T cells which were obtained or harvested from a geriatric donor.

“Young donor” as used herein refers to a human less than 55 years, 50 years or 45 years of age. “Young T cell” as used herein refers to T cells which were obtained or harvested from a young donor.

The terms “patient,” “subject,” “individual,” and the like are used interchangeably herein, and each can refer to any animal (e.g., rodents such as mice and rats), or cells thereof whether in vitro or in situ, amenable to the methods described herein. In certain non-limiting embodiments, the patient, subject or individual is a human or a non-human primate.

As used herein, the expression “specifically binds” in reference to a chimeric T cell receptor means that the chimeric T cell receptor binds to its target protein with greater affinity that it does to a structurally different protein(s).

### II. Geriatric Vs. Young T Cells

Given the rapid development of chimeric antigen receptor T cell (CAR-T) therapies in the context of an aging population that is at high risk for cancer, a study was performed to address potential age-related CAR-T cell deficiencies. It was speculated that CAR-T produced from geriatric donors would demonstrate functional impairments relative to CAR-T from younger individuals or young donors. Furthermore, a mechanism was postulated for geriatric CAR-T (gCAR-T) impairment and a strategy for improving the efficiency of generating of gCAR-T having therapeutic activity in vitro or in vivo.

Generating CAR-T involves first harvesting cells such as peripheral mononuclear cells (PBMCs) from a subject, usually a subject suffering from a cancer and in need of medical intervention to treat the cancer. It was determined by the experiments described herein, including Example 1, that T cells obtained from geriatric donors suffered from reduced transduction by viral vectors which harbored recombinant nucleic acid molecules encoding CAR constructs as compared to transduction of T cells from young donors. To examine the differences between the transduction efficiency of the geriatric and young donor T cells, PBMC was isolated from both young and geriatric donors and activated using methods standard in the art followed by retroviral transduction to generate CAR-T cells specific for CEA. Transduction efficiency was measured using cytometric flow analysis and was found to be significantly lower in gCAR-T cells (CAR-T cells from geriatric donors) compared to yCAR-T cells (cells from young donors). It's known that aging causes a decrease in lymphocyte proliferative capacity due to changes in cell surface markers and signal transduction pathways that get altered due to ageing (Fulop et al., 2007, Clin Interv Aging, 2:33-54).

Moreover, when both untransduced and transduced cells were maintained in IL2 or IL15 to allow proliferation and maintenance of desired CAR-T cells, there was significantly higher expansion of both untransduced and transduced yCAR-T cells as compared to gCAR-T cells. It is known to the ordinarily skilled artisan that the IL2/IL15Rβγc receptors activate multiple proliferation pathways that include Akt, Erk and Jak-dependent activation of Stat3 and Stat5 (Cornish et al., 2006, Blood, 108:600-608). Notably, phosphorylation of Akt, Erk, Stat3 and Stat5 was shown to be significantly increased in yCAR-T cells compared to gCAR-T cells indicating that the gCAR-T cells had an impaired pro-proliferation signaling despite being treated with proliferation cytokines like IL2 and IL15.

T cell receptor (TCR) along with CD3 activation induces an intricate signaling cascade which leads to naïve T cell proliferation and differentiation into specific effector cells (Brownlie and Zamoyska, 2013, Nat Rev Immunol, 13:257-269). This differentiation depends on the cytokine milieu and on other factors including but not limited to antigens, antigen presenting cell types and co-stimulatory molecules. Studies show the importance of the persistence of memory CD8+ T cells which are known to recognize and destroy tumor cells (Wrzesinski and Restifo, 2005, Curr Opin Immunol, 17:195-201). Also the presence of memory T cell phenotype helps preventing tumor relapse (Straetemans et al., 2015, Mol Ther, 23:396-406). The phenotypic differences among CD4 and CD8 CAR-T between the young and geriatric groups was studied. Phenotypic profiling was performed by using CD45RA+CD45RO-CCR7+CD62L+ for naïve cells, CD45RA-CD45RO+CCR7+CD62L+ for central memory cells, CD45RA-CD45RO+CCR7-CD62L− for effector memory cells and CD45RA+CD45RO-CCR7-CD62L− for effector cells (Sallusto et al., 2004, Annu Rev Immunol, 22:745-763). All cell populations were gated as CD3+CAR+ and then gated either of CD4+ or CD8+. The data showed that yCAR-T contain higher proportions of CD4 effector cells (EC), CD4 effector memory (EM) cells and CD8 effector memory cells (EM). No significant differences were observed in the phenotypes between the IL2 and IL15 treated cells. Activated T cells proliferate and differentiate into CM and EC. EC and CM can further differentiate to EM cells. Naïve, effector, CM, and EM exit LN from EL. (Obharai et al., 2006, 176:4051-4058) EM provides immediate protection from a peripheral challenge while TCM provide protection from systemic challenge and generate effector cells (Bouneaud et al., 2005, J Exp Med, 201:579-590). Human CD8 TEM exhibit direct ex vivo lytic activity and constitutively express granzymes and perforin, and hence are likely derived from effector cells (Marzo et al., 2007, J Immunol, 179:36-40).

To evaluate the anti-tumor efficacy of the CAR-T cells, cytotoxicity assays were performed in which CAR-T cells were incubated with mouse colon carcinoma cells which expressed CEA (CEA+) or with MC38 cells that were CEA- to be used as a negative control and to show activity was specific to the target CEA molecule bound by the CAR-T receptor protein. The gCAR-T cells which had been expanded and maintained in either IL2 or IL15 had significantly lower CEA specific cytotoxicity, however, there was no difference in untransduced CAR-T cells which had been expanded and maintained in IL2 or IL15. Cytotoxic T cells lyse target cells in this case tumor cells via granzyme/perforin (Sin et al., 2010, Cancer Immunol Immunother, 61:1671-1682). It was subsequently determined in the present studies that yCAR-T cell populations had increased numbers of CD4+ effector cells as compared to the gCAR-T cell populations. Accordingly, in some embodiments, gCAR-T cells populations which are treated to enhance or increase the number or relative number of CD4+ cells are disclosed herein, wherein the treated gCAR-T cells has cytotoxicity levels which are greater than comparable gCAR-T cells which were not treated in a manner to increase the number or relative number of CD4+ cells. Importantly, when the cytotoxicity was normalized for the transduction efficiency which is lower for gCAR-T cells the values were not significantly different from the yCAR-T cells. Accordingly, also provided herein are methods for increasing the transduction efficiency of cells, e.g. T cells, obtained from a subject. In some embodiments, the subject is a geriatric patient.

### III. Rescuing Geriatric T Cells

Integrins belong to cell surface adhesion molecule family that mediate cell adhesion and are responsible for cell-cell adhesion and cell migration (Weber et al., 2011, J Cell Sci, 124:1183-1193). α5β1 integrins play a crucial role in retronectin based transduction of T cells (Yu et al., 2008, Cancer Gene Ther, 15:508-516). Retronectin, a recombinant human fibronectin fragment contains a cell binding domain which binds mainly by interacting with the α5β1 integrins of the cells and thereby aids in co-localization of the cells with the viral particle. As shown in Example 3 below, significantly higher expression of α5β1 integrins were observed in the young donor T cells as compared to the geriatric donor T cells. This increased expression correlates with lower gCAR-T transduction efficiencies. M-CSF and TGF-β1 are known to up-regulate α5β1 integrin expression and enhance cell adhesion and migration (Cai et al., Biochem Biophys Res Commun, 274:519-525, Shima et al., 1995, Proc Natl Acad Sci USA, 92:5179-5183). They are also known to modulate several different integrins such as α1, α2, α3, α5 and β1 on many different cell types (chondrocyte, breast cancer cell, fibroblast, osteoblast, colon carcinoma, etc.) (Hynes, 2002, Cell, 110:673-687). Accordingly studies were done (e.g., Example 3) to increase expression of α5β1 integrin expression levels in geriatric T cells. PBMC isolated from geriatric donors were treated with either M-CSF or TGFβ1. As a result, a significant increase of the α5β1 integrin expression in geriatric cells treated with either M-CSF or TGFβ1 was observed. Moreover, the expression of α5β1 in the geriatric cells was comparable to that of the young T cells.

To show that the increase in α5β1 expression is correlated with an increase in transduction efficiency, transductions of both the geriatric and young donor T cells treated with M-CSF or TGFβ1 were transduced at comparable transduction efficiencies (Example 3). Moreover, evaluation of the anti-tumor efficiency of the treated gCAR-T cells using cytotoxicity assays demonstrated that treatment of gCAR-T cells with either M-CSF or TGF-β1 increased the cell-killing activity of the gCAR-T cell population.

In conclusion, this study suggests that impaired CAR expression among gCAR-T can hinder anti-tumor efficacy of CEA specific CAR-T cells which can cause a huge impediment in immunotherapy of geriatric cancer patients. Our pre-clinical in vitro data suggests that this phenotype can be reversed with treating the geriatric T cells with M-CSF or TGF-β1 to rescue the anti-tumor activity of these compromised cells. Confirmation of these findings in other CAR-T products in addition to in vivo testing is required prior to clinical application. This study has translational impact as it suggests that age-related CAR-T deficiencies are reversable.typing here! (If you reach 100, begin using ALT+2)

### IV. EXAMPLES

The following examples are illustrative in nature and are in no way intended to be limiting.

### Example 1: Generating Anti-CEA CAR-T Cells

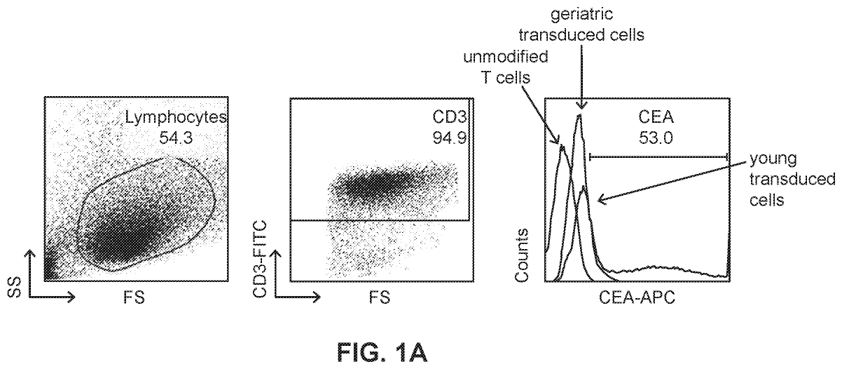

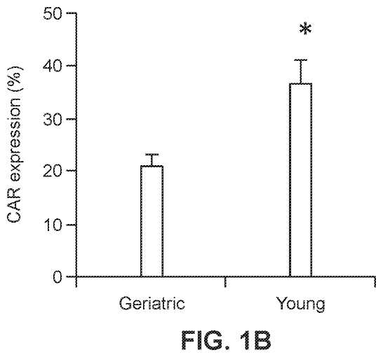

To examine the differences between the transduction efficiency of geriatric T cells and young donor T cells, PBMC was isolated from the whole blood obtained from 8 geriatric patients greater than 65 years of age (gCAR-T group) and from 8 non-geriatric patients ranging in age from 18 to 45 years (yCAR-T group). The PBMC from each group were activated for 48 hrs with OKT3 (50 ng/ml, Ortho Biotech) and cells were maintained at a concentration of 2×10⁶/ml. The PMBC were transduced 3 times using a retroviral delivery system containing the tandem molecule generated by molecular fusion of hMN14 sFv-CD8 hinge segment of the IgTCR (IgCEA) in the MFG retroviral backbone with a hybrid CD28/CD3 molecule (generated according to the methods of Emtage et al., 2008, Clin Cancer Res, 14:8112-8122). Transduction efficiency was measured 48 hrs post-transduction using a CEA-Ig fusion protein which is specifically bound by surface-expressed anti-CEA CAR construct. Binding of the CEA-Ig fusion protein by transduced cells was detected using flow cytometry. A standard gating strategy was used to identify viable, single cells expressing CD3 and the chimeric anti-CEA CAR-T. The results, presented in FIG. 1A, show that viral transduction efficiency was significantly lower in cells obtained from the gCAR-T group (21.3%) than in cells obtained from the yCAR-T group (36.9%) with n=8, p=0.002 (FIG. 1B).

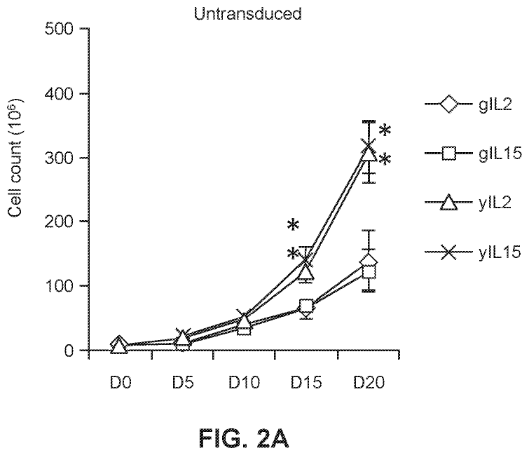

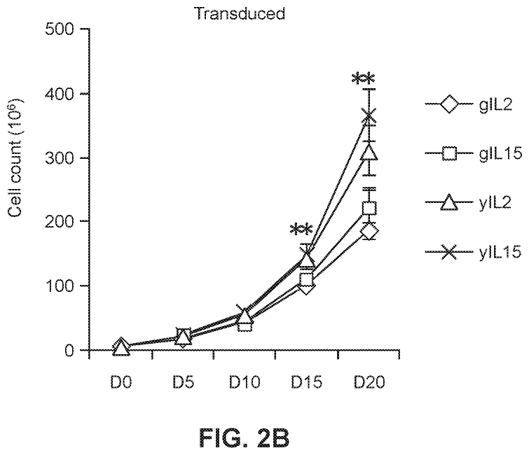

Untransduced and transduced cells from each of the two Groups were maintained in IL2 (3000 IU/ml) or IL15 (5 ng/ml) for a period of 20 days and growth curves were measured. There was significantly higher expansion of both untransduced (FIG. 2A) and transduced (FIG. 2B) yCAR-T cells compared to expansion of gCAR-T cells for both IL2 and IL15. Expansion of both untransduced and transduced yCAR-T cells was ˜2.5 fold higher than untransduced and transduced gCAR-T cells (FIGS. 2A and 2B). Results are shown as mean±SEM (*p<0.05).

### Example 2: Molecular Profiles of yCAR-T and gCAR-T Cells

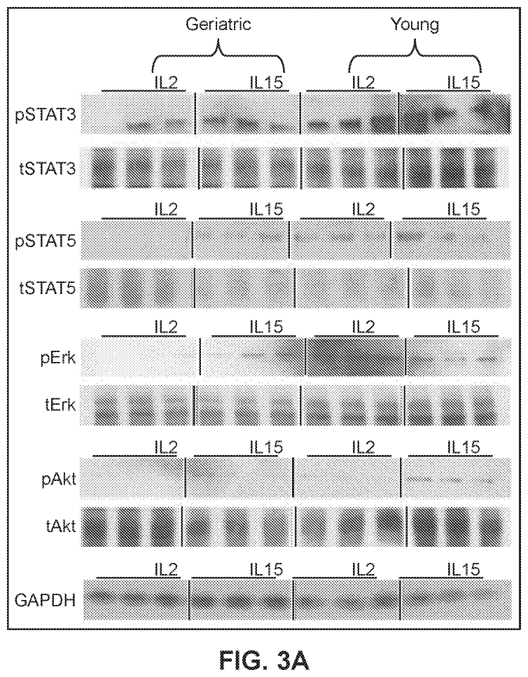

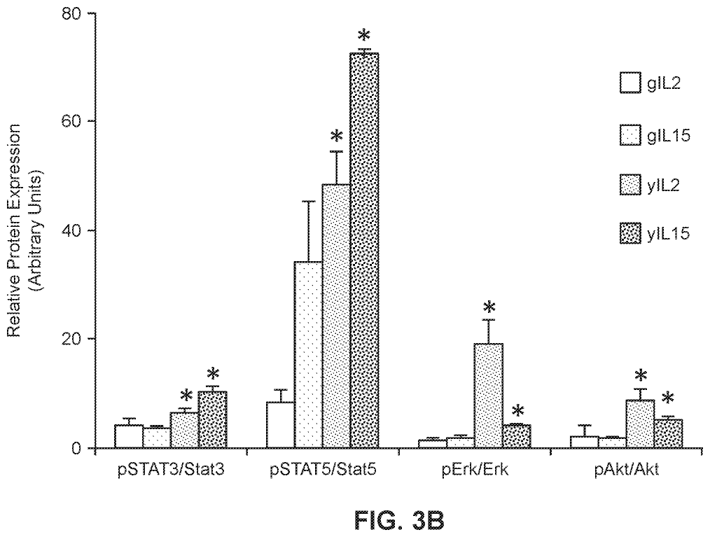

The IL2/IL15Rβγc receptors activate multiple proliferation pathways that include Akt, Erk and Jak-dependent activation of Stat3 and Stat5 (Cornish et al., 2006, Blood, 108:600-608). To further understand differences between yCAR-T and gCAR-T cells and function, experiments were performed to measure phosphorylation of Stat3, Stat5, Erk and Akt in yCAR-T and gCAR-T cells expressing the anti-CEA CAR. Total protein was isolated on day 20 from gCAR-T and yCAR-T cells generated and maintained as described in Example 1 and treated with IL2 or IL15. Cells were washed twice with ice-cold PBS and lysed with RIPA buffer (Life Technologies) supplemented with protease inhibitor cocktail (Roche Diagnostics), 1 mM NaVO4, and 1 mM NaF as desacribed in Ghosh et al., 2012, Proc Natl Acad Sci, 109:10024-10029). Lysates were centrifuged at 10,000 rpm for 10 min at 4° C., and supernatants were collected and protein quantification was performed using the Bradford protein assay (Thermo Scientific) with BSA as the standard. Lysates were denatured using (3-mercaptoethanol (Life Technologies) and Laemmli sample buffer (Bio-Rad) and heated at 70° C. for 10 min, electrophoresed in Mini Protean TGX4-15% gels (Biorad), transferred to TransBlot Turbo PVDF membrane (Biorad) and immunoblotted with primary antibodies against phosphorylated and total Erk, Akt, Stat3 and Stat5 proteins. Primary antibody binding was detected using HRP-conjugated secondary antibodies (Santa Cruz) and ECL Prime Wetser Blot reagents (Amersham) as chemiluminescence substrates. The immunoblots were reprobed with antibody against GAPDH to control for equal loading. Triplicate samples were loaded for each treated group and the signals were quantified by denstimotric analysis and normalized to respective total proteins. Phosphorylation of each of Akt, Erk, Stat3 and Stat5 was significantly increased in yCAR-T cells as compared to the gCAR-T cells (FIGS. 3A and 3B) indicating that the gCAR-T cells had an impaired pro-proliferation signaling despite being treated with proliferation cytokines such as IL2 and IL15.

### Example 3: In Vitro Anti-Tumor Activity of CAR-T Cells

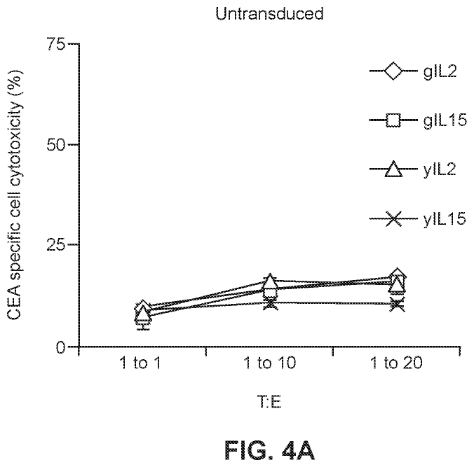

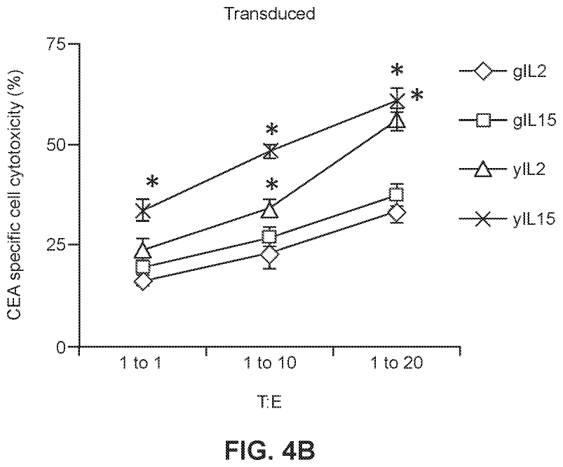

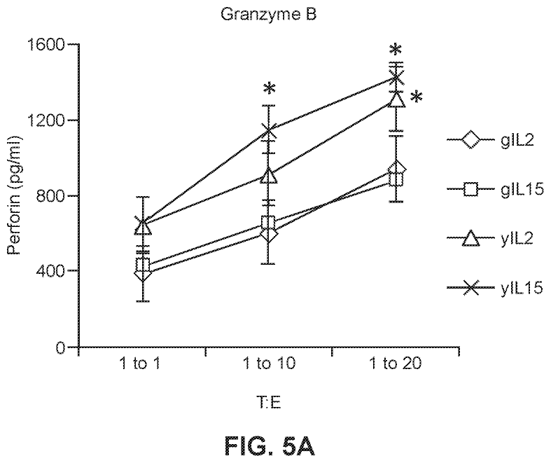

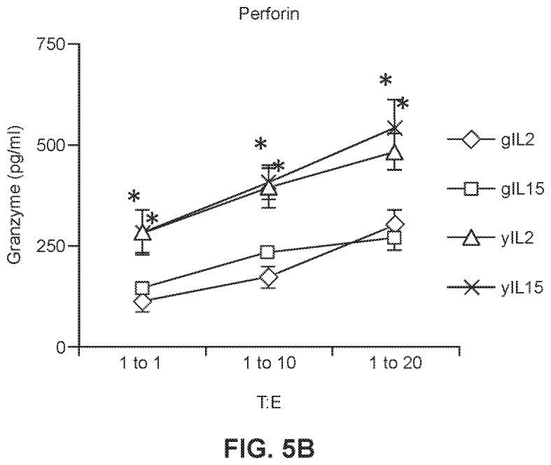

To look at the anti-tumor efficacy of the anti-CEA young and geriatric CAR-T cells, cytotoxicity assays were performed in which the anti-CEA CAR-T cells described in Example 1 were incubated with MC38 (murine colon carcinoma cell line) target cells. The y-CAR-T and gCAR-T cells (effector cells (E)) were treated with IL2 (3000 IU/ml) or IL15 (5 ng/ml) as described in Example 1, then incubated with MC38CEA+(target cells (T)) cells with 1:1, 1:10 and 1:20 target to effector cell ratios. MC38 cells are stably transfected with a gene encoding human CEA to generate the MC38CEA+ cells. MC38 CEA− cells were used as a negative control to show CEA-specific cytotoxicity of CAR-T cells. Cell lysis (cytotoxicity) was determined by measuring the release of LDH (lactose dehydrogenase) into the cell culture supernatant. As shown in FIGS. 4A and 4B, the gCAR-T cells for both IL2 and IL15 treated gCAR-T had significantly lower CEA specific cytotoxicity than IL2 and IL15 treated yCAR-T cells (˜2 fold less than yCAR-T, p<0.005) with no difference in untransduced CAR-T cells. Secretion of perforin and Granzyme B by IL2- or IL15-treated yCAR-T and gCAR-T cells was also measured. As shown in FIGS. 5A and 5B, secretion of Granzyme B (FIG. 5A) and perforin (FIG. 5B) by yCAR-T cells was significantly greater than secretion by the gCAR-T cells. Results are shown as mean±SEM. *p<0.05. Enhanced tumor killing by yCAR-T correlated with increased levels of perforin (1.3±0.1 ng/ml, p=0.01) and granzyme B (0.4±0.03 ng/ml, p=0.02) as compared to gCAR-T for both IL2 and IL15 treatments. When the cytotoxicity was normalized for the transduction efficiency, which is lower for gCAR-T cells, the values were not significantly different between the gCAR-T cells and the yCAR-T cells (data not shown).

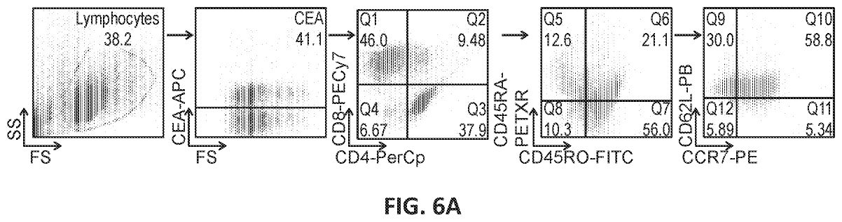

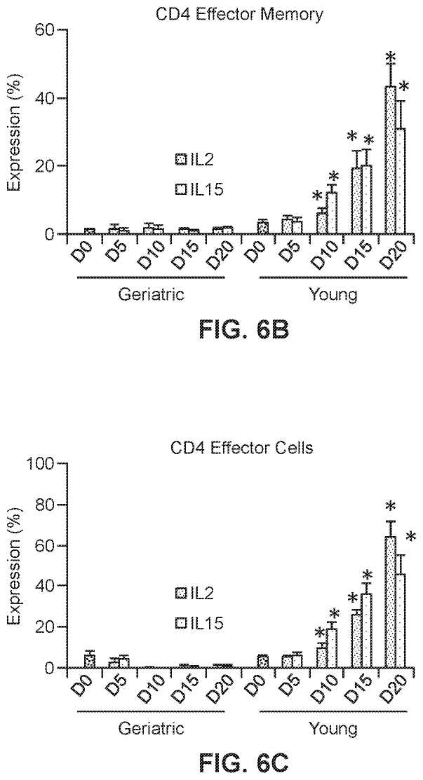

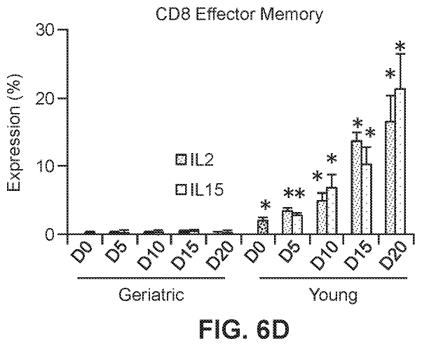

CD4 or CD8 memory and effector T cell phenotypic analysis was performed based on CD45RA, CD45RO, CD62L and CCR7 expression (n=8 for each of the yCAR-T and gCAR-T cells) using the gating strategy. CEA specific CD4 and CD8 T cells were phenotyped for effector, central memory and effector memory cells. The phenotypic differences among CD4 and CD8 CAR-T between the yCAR-T and gCAR-T cells was examined. Phenotypic profiling was performed by using CD45RA+CD45RO-CCR7+CD62L+ for naïve cells, CD45RA-CD45RO+CCR7+CD62L+ for central memory cells, CD45RA-CD45RO+CCR7-CD62L− for effector memory cells and CD45RA+CD45RO-CCR7-CD62L− for effector cells (Sallusto et al., 2004, Annu Rev Immunol, 22:745-763). All cell populations were gated as CD3+CAR+ and then gated either of CD4+ or CD8+ as shown in FIG. 6A. FIGS. 6B, 6C and 6D show that as compared to gCAR-T cells, yCAR-T cells contained higher proportions of CD4 effector memory (EM) cells (43.2± 6.8%, p=0.002), CD4 effector cells (EC) (64.3±7.6%, p=0.003), and CD8 effector memory cells (EM) (16.5±3.6%, p=0.002). Results are shown as mean±SEM. *p<0.05 vs. geriatric donor T cells for respective groups (IL2 or IL15). No significant differences in the phenotypes between the IL2 and IL15 treated cells were observed.

Activated T cells proliferate and differentiate into CM T cells and EC. EC and CM cells can further differentiate to EM cells. Naïve, effector, CM, and EM cells exit LN cells from EL cells. (12) EM cells provide immediate protection from a peripheral challenge while TCM provide protection from systemic challenge and generate effector cells (Bouneaud et al., 2005, J Exp Med, 201:579-590). Human CD8 TEM exhibit direct ex vivo lytic activity and constitutively express granzymes and perforin, and hence are likely derived from effector cells (Marzo et al., 2007, J Immunol, 179:36-40)

As such, further experiments were focused on rescuing gCAR-T transduction. Cytotoxic T cells lyse target cells in this case tumor cells via granzyme/perforin. We found that there was enhanced numbers of CD4+ effector cells in yCAR-T cells as compared to gCAR-T cells and possibly this was causing the increased cytotoxicity observed in that group.

### Example 4: Rescuing gCAR-T Cells

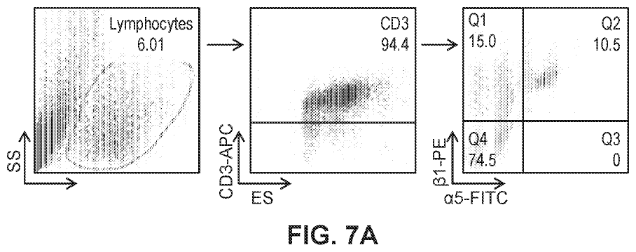

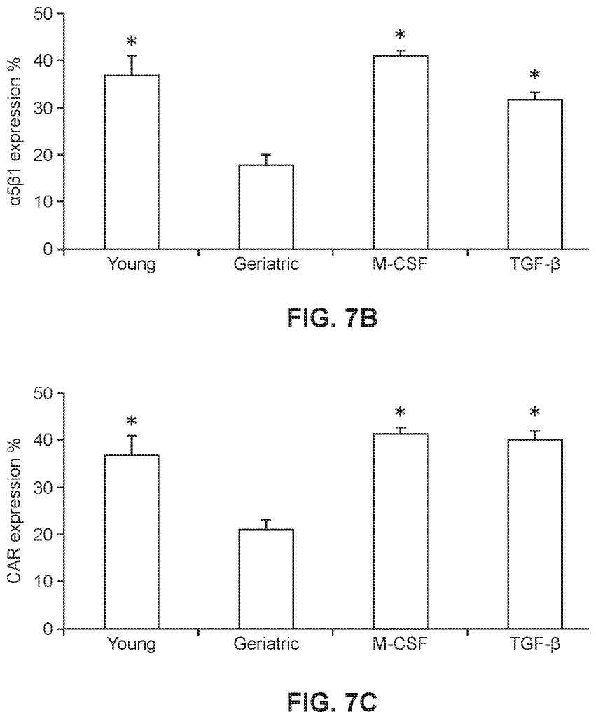

In an effort to rescue the decreased α5β1 integrin expression levels in geriatric T cells, and thereby increase viral transduction, PBMC isolated from geriatric donors were treated with either M-CSF (5 ng/ml) or TGFβ1 (10 ng/ml) and effects of the treatments on α5β1 integrin expression in the CAR-T cells were measured. Flow cytometric analysis was performed (FIG. 7A) and the relative expression of α5β1 integrin in M-CSF and TGFβ1 treated activated geriatric T cells were quantified in all 8 donors and compared to untreated yCAR-T and gCAR-T cells (n=8 for each group). A significant increase of the α5β1 integrin expression in gCAR-T cells was observed for both the M-CSF and TGFβ1 treatments. The α5β1 integrin expression in gCAR-T cells, shown in FIG. 7B, was comparable to α5β1 integrin expression in the yCAR-T cells (gCAR-T cells+M-CSF: 41.0+1.0, gCAR-T cells±TGFβ1: 31.8±1.4, yT cells 44.41±4.2, p<0.0005). The treated cells were then transduced with the retrovirus containing the anti-CEA CAR-T construct as performed in Example 1 and the transduction efficiency increased in the M-CSF and TGFβ1 treated gCAR-T cells (yCAR-T 36.9±4.3, gCAR-T 29.9±1.0, gCAR-T+M-CSF 41.4±1.0, gCAR-T±TGFβ1 40.1±2.0, p<0.002 vs gCAR-T (FIG. 7C).

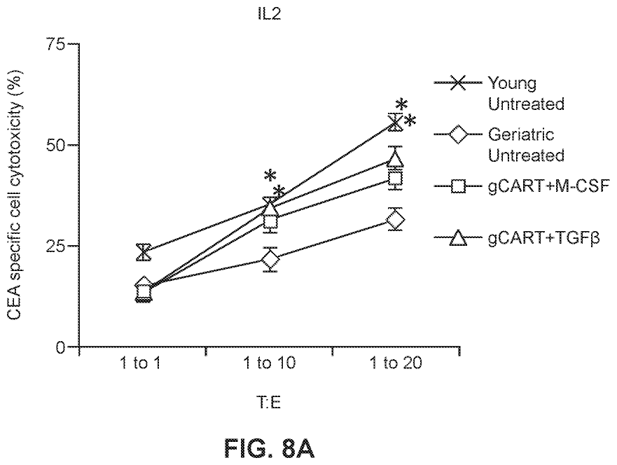

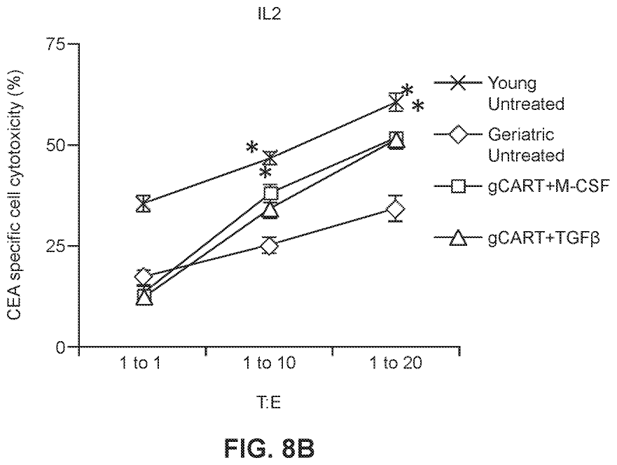

The anti-tumor cytotoxic activity of the treated gCAR-T cells was then evaluated as in Example 2. Cytotoxicity assays were performed by incubating MC38CEA+ cells with yCAR-T cells and gCAR-T cells that had been treated with either IL2 (FIG. 8A) or IL15 (FIG. 8B). The yCAR-T and gCAR-T effector cells were evaluating by incubating them with the MC38 CEA+ target cells at a target to effector cell ratio of 1:1, 1:10 and 1:20. Control experiments were also performed using MC38 CEA− cells as a negative control to show CEA specificity of the CAR-T cells. After analysis of the data the results showed that for both IL2 and IL15-treated cells, treatment of gCAR-T cells with M-CSF or TGFβ1 appeared to have rescued the gCAR-T cells with respect to cytotoxicity. Specifically, gCAR-T cells treated with either M-CSF (FIG. 8A, results are shown as mean±SEM. *p<0.005 vs. untreated gCAR-T cells) or TGFβ1 (FIG. 8B, results are shown as mean±SEM. *p<0.0005 vs. untreated gCAR-T cells) had significantly increased cytotoxic activity compared to gCAR-T cells untreated with either M-CSF or TGFβ1.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

1. A method for transducing a population of lymphocytes ex vivo, comprising
contacting ex vivo the lymphocytes with an agent which increases expression of α5β1 by the lymphocytes; and
mixing the lymphocytes with a recombinant viral particle which comprises a recombinant DNA molecule encoding a chimeric antigen receptor (CAR).

2. The method according to claim 1, wherein the contacting the lymphocytes with the agent comprises incubating the lymphocytes with the agent.

3. The method according to claim 1, wherein the agent comprises M-CSF or TGβ1.

4. The method according to claim 1, wherein the contacting the lymphocytes with the agent comprises incubating the lymphocytes with 0.1 ng/ml to 10 ng/ml, 2.5 ng/ml to 7.5 ng/ml or 4 ng/ml to 7 ng/ml M-CSF.

5. The method according to claim 1, wherein the contacting the cells with the agent comprises incubating the cells with 1 ng/ml to 20 ng/ml, 5 ng/ml to 15 nm/ml, or 7.5 ng/ml to 12.5 ng/ml TG931.

6. The method of claim 1, wherein prior to contacting the cells with the agent the cells are obtained from a subject diagnosed with a disease.

7. The method of claim 6, wherein the disease is a cancer or an acquired immunodeficiency disease.

8. The method of claim 7, wherein the cancer is selected from the group consisting of a liver cancer, a pancreatic cancer, a leukemia, a lymphatic cancer, a brain cancer, a head & neck cancer, a lung cancer, a breast cancer, a thyroid cancer, a prostate cancer, a stomach cancer, an esophageal cancer, a colon cancer, a rectal cancer, a testicular cancer, a bladder cancer, a cervical cancer, an ovarian cancer and a skin cancer.

9. The method of claim 8, wherein the disease is a cancer or an acquired immunodeficiency disease.

10. The method of claim 1, wherein the recombinant viral particle is a lentiviral, retroviral, adenoviral or adeno-associated viral vector.

11. A method for treating a subject in need thereof comprising administering to the subject a composition comprising a CAR-T generated using the method according to claim 1.

12. The method according to claim 11, wherein the subject is at least 65 years old.
